(19) United States
(12) Patent Application Publication
(10) Pub. No.: US 2026/0347924 A1
(43) Pub. Date: Nov. 5, 2026

(54) Systems and Methods for Dual-End Sequencing

(71) Applicant: Pacific Biosciences of California, Inc., Menlo Park, CA (US)

(72) Inventors: Lindsey Williams, Livemore, CA (US); Chandan Shee, Newark, CA (US); Bojan Berghuis, Burlingame, CA (US); Bryan P. Staker, Pleasanton, CA (US); Abizar Lakdawalla, Seattle, WA (US)

(73) Assignee: Pacific Biosciences of California, Inc., Menlo Park, CA (US)

(21) Appl. No.: 18/994,456

(22) PCT Filed: Jul. 18, 2023
(86) PCT No.: PCT/US2023/070442

§ 371 Date: Jan. 14, 2025

Related U.S. Application Data
(60) Provisional application No. 63/391,664, filed on Jul. 22, 2022.

(51) Int. Cl. C12Q 1/6869; C12N 9/22; C12Q 1/48; C12Q 1/6806; C12Q 1/6813; C12Q 1/6855; C12Q 1/6876

(52) U.S. Cl. CPC C12Q 1/6869; C12N 9/222; C12Q 1/48; C12Q 1/6806; C12Q 1/6813; C12Q 1/6855; C12Q 1/6876; C12Y 201/01; C12Y 207/07

(57) ABSTRACT

Provided are systems and compositions for dual-end sequencing.

### CROSS-REFERENCE

This application is a U.S. national phase application under 35 U.S.C. § 371 of International Application No. PCT/US2023/070442, filed Jul. 18, 2023 and published as WO 2024/020410 on Jan. 25, 2024, which claims the benefit of U.S. Provisional Application No. 63/391,664, filed Jul. 22, 2022, which application is incorporated herein by reference.

### SUMMARY

In certain aspects, described herein is a method of preparing a double-stranded sample nucleic acid for sequencing, wherein the double-stranded sample nucleic acid comprises a forward sample strand and a reverse sample strand, the method comprising: (a) contacting the sample nucleic acid with one or more first adaptors to form a sample-adaptor complex, wherein the one or more first adaptors prevent ligation between the 5′ end of the forward sample strand and the 5′ end of the reverse sample strand; (b) extending with a polymerase to form a forward double-stranded sample-adaptor complex comprising the forward strand, the first adaptor, and a sequence complementary to the forward strand and a reverse double-stranded sample-adaptor complex comprising the reverse sample strand, the first adaptor and a sequence complementary to the reverse sample strand; and (c) ligating one or more second adaptors to the forward double-stranded sample-adaptor complex and the reverse double-stranded sample-adaptor complex to form a circular forward-adaptor complex and a circular reverse-adaptor complex. In some embodiments, the forward double-stranded sample-adaptor complex and the reverse double-stranded sample-adaptor complex produced in step (b) comprise a blunt end and a looped end. In some embodiments, the first adaptor comprises a first primer hybridization site, and the second adaptor comprises a second primer hybridization site. In some embodiments, the first primer hybridization site and the second primer hybridization site are different. In some embodiments, the first adaptor and the second adaptor are different. In some embodiments, the first adaptor comprises: a first nucleic acid strand comprising: a 3′ segment of the first nucleic acid strand comprising a self-complementary stem and a loop, and a 5′ segment of the first nucleic acid strand comprising a sequence that is not self-complementary; and a second nucleic acid strand comprising:, a 3′ segment of the second nucleic acid strand comprising a sequence complementary to a portion of the 5′ segment of the first nucleic acid strand and a 5′ segment that blocks s ligation of the 3′ end of the first nucleic acid strand to the 5′ end of the second nucleic acid strand, thereby generating a non-contiguous segment between the 5′ end of the second nucleic acid strand and the 3′ end of the first nucleic acid strand, wherein the 5′ segment comprises a flap or a blocking group. In some embodiments, the first adaptor prevents ligation by a gap, a flap, or a blocking group. In some embodiments, the second adaptor comprises a hairpin. In some embodiments, the method further comprises contacting the circular forward-adaptor complex or the circular reverse-adaptor complex with a methyltransferase enzyme.

In certain aspects, described herein is a method, comprising: (a) providing a double-stranded nucleic acid molecule comprising a first strand and a second strand hybridized to the first strand; (b) ligating adaptors to ends of the double-stranded nucleic acid molecule to yield a circular nucleic acid molecule; and (c) using the circular nucleic acid molecule as a template to generate a single-stranded nucleic acid molecule comprising copies of the first strand and the second strand. In some embodiments, the first adaptor is ligated to a first end of the double-stranded nucleic acid molecule before a second adaptor is ligated to a second end of the double-stranded nucleic acid molecule. In some embodiments, the double-stranded nucleic acid molecule is immobilized when the first adaptor is ligated to the first end of the double-stranded nucleic acid molecule. In some embodiments, a first adaptor and a second adaptor are simulatanously ligated. In some embodiments, the method further comprises selecting a circular nucleic acid molecule wherein the first adaptor and the second adaptor are different. In some embodiments, step (b) comprises: ligating Y-adaptors to ends of the double-stranded nucleic acid molecule to produce an adaptor-nucleic acid complex; amplifying the adaptor-nucleic acid complex to produce a nucleic acid complex with a first adaptor sequence at a first end and a second adaptor sequence at a second end; and ligating the 5′ end of the first adaptor sequence with the 3′ end of the first adaptor sequence and ligating the 5′ end of the second adaptor sequence with the 3′ end of the second adaptor sequence to produce a circule nucleic acid molecule. In some embodiments, the first adaptor comprises a first primer hybridization site, and the second adaptor comprises a second primer hybridization site. In some embodiments, the first primer hybridization site and the second primer hybridization site are different. In some embodiments, the method further comprises contacting the single-stranded nucleic acid molecule with a methyltransferase enzyme.

In certain aspects, described herein is a method of paired end clonal amplification comprising: (a) contacting a sample double-stranded DNA molecule comprising a template sequence with a first adaptor comprising a first adaptor sequence and a second adaptor comprising a second adaptor sequence to produce a circular DNA molecule, wherein the first adaptor sequence comprises a first primer hybridization sequence and wherein the second adaptor sequence comprises a second primer hybridization sequence; (b) contacting the circular DNA molecule with a polymerizing enzyme to produce a single-stranded DNA molecule via rolling circle amplification, wherein the single-stranded DNA molecule comprises a sequence comprising at least the first adaptor sequence, the template sequence, the second adaptor sequence, and a sequence complementary to the template sequence; and (c) subjecting the single-stranded DNA molecule to clonal amplification from at least (i) the first primer hybridization sequence and (ii) the second primer hybridization sequence. In some embodiments, subjecting the single stranded DNA molecule to clonal amplification from (i) the first primer hybridization sequence comprises contacting the ssDNA with a blocking molecule that prevents extension of a nascent sequence beyond the second adaptor; wherein the nascent sequence is complementary to the template sequence or the sequence complementary to the template sequence. In some embodiments, subjecting the single stranded DNA molecule to clonal amplification from (ii) the second primer hybridization sequence comprises contacting the ssDNA with a blocking molecule that prevents extension of a nascent sequence beyond the first adaptor; wherein the nascent sequence is complementary to the template sequence or the sequence complementary to the template sequence. In some embodiments, the blocking molecule comprises an oligonucleotide. In some embodiments, the oligonucleotide comprises a locked nucleic acid (LNA), a psoralen modified nucleic acid, a MGB modified nucleic acid, or a G-quadruplex oligo. In some embodiments, the blocking molecule comprises a peptide. In some embodiments, the peptide comprises a sequence-specific DNA binding protein. In some embodiments, the sequence-specific DNA binding protein is a Cas protein or a Tus protein. In some embodiments, the clonal amplification in (c)(i) is performed before the clonal amplification in (c)(ii). In some embodiments, the clonal amplification in (c)(ii) is performed before the clonal amplification in (c)(i). In some embodiments, the single-stranded DNA molecule further comprises a second copy of the first adaptor sequence, a second copy of the sequence of interest, a second copy of the second adaptor sequence, and a second sequence complementary to the sequence of interest. In some embodiments, step (a) occurs in solution. In some embodiments, step (b) occurs in solution. In some embodiments, step (c) occurs in a solid state. In some embodiments, the first adaptor is ligated to the double-stranded DNA molecule before the second adaptor is ligated to the double-stranded DNA molecule. In some embodiments, the first adaptor comprises: a first nucleic acid strand comprising: a 3′ segment of the first nucleic acid strand comprising a self-complementary stem and a loop, and a 5′ segment of the first nucleic acid strand comprising a sequence that is not self-complementary; and a second nucleic acid strand comprising:, a 3′ segment of the second nucleic acid strand comprising a sequence complementary to a portion of the 5′ segment of the first nucleic acid strand and a 5′ segment that blocks s ligation of the 3′ end of the first nucleic acid strand to the 5′ end of the second nucleic acid strand, thereby generating a non-contiguous segment between the 5′ end of the second nucleic acid strand and the 3′ end of the first nucleic acid strand, wherein the 5′ segment comprises a flap or a blocking group. In some embodiments, the circular DNA molecule comprises either the forward strand of the sample double-stranded DNA molecule or the reverse strand of the sample double-stranded DNA molecule, but not both. In some embodiments, the first adaptor and the second adaptor are ligated to the double-stranded DNA molecule simultaneously. In some embodiments, the circular DNA molecule comprises the forward strand of the sample double-stranded DNA molecule and the reverse strand of the sample double-stranded DNA molecule.

In certain aspects, described herein is an adaptor, comprising: a first nucleic acid strand comprising: a 3′ segment of the first nucleic acid strand comprising a self-complementary stem and a loop, and a 5′ segment of the first nucleic acid strand comprising a sequence that is not self-complementary; and a second nucleic acid strand comprising:, a 3′ segment of the second nucleic acid strand comprising a sequence complementary to a portion of the 5′ segment of the first nucleic acid strand and a 5′ segment that blocks ligation of the 3′ end of the first nucleic acid strand to the 5′ end of the second nucleic acid strand, thereby generating a non-contiguous segment between the 5′ end of the second nucleic acid strand and the 3′ end of the first nucleic acid strand, wherein the 5′ segment comprises a flap or a blocking group. In some embodiments, the adaptor comprises DNA. In some embodiments, the adaptor comprises a first sequencing site. In some embodiments, the 5′ segment of the second nucleic acid comprises a flap of greater or equal to 1 nucleotides. In some embodiments, the 5′ segment of the second nucleic acid comprises a blocking group. In some embodiments, the blocking group is an oligonucleotide or a sequence for binding a sequence-specific DNA binding protein. In some embodiments, the oligonucleotide comprises a locked nucleic acid (LNA), a psoralen modified nucleic acid, a MGB modified nucleic acid, or a G-quadruplex oligo. In some embodiments, the sequence-specific DNA binding protein comprises a Cas protein or a Tus protein.

In certain aspects, described herein is a DNA molecule comprising: a first DNA strand comprising: a first segment comprising a first sequence; wherein the first segment is ligated to a first hairpin segment comprising a first hairpin sequence at the 3′ end of the first hairpin segment; a second segment comprising a second sequence that is complementary to the first sequence; wherein the second segment is ligated to the first hairpin segment at the 5′ end of the second segment and wherein the second segment is ligated to a second hairpin segment comprising a second hairpin sequence at the 3′ end of the second segment; a third segment comprising the first sequence, wherein the third segment is ligated to the second hairpin sequence at the 5′ end of the third segment and wherein the third segment is ligated to a third hairpin segment comprising the first hairpin sequence at the 3′ end; a fourth segment comprising the second sequence; wherein the fourth segment is ligated to the third hairpin segment at the 5′ end of the fourth segment; and a second DNA strand comprising a sequence complementary to the first sequence.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a sample DNA ligated to 2 different adaptors.

FIG. 2 depicts a single-stranded circular DNA formed by ligating two adaptors to a double-stranded DNA.

FIG. 3 depicts a single-stranded concatemer produced by rolling circle amplification.

FIG. 4 depicts the process of rolling circle amplification while a methyltransferase is present.

FIG. 5 depicts a methylated concatemer.

FIG. 6 depicts sequencing a methylated concatemer.

FIG. 7 depicts sequencing a methylated concatemer after bisulfite conversion.

FIGS. 8A-8B depict a method of sequencing using asymmetric adaptors.

FIG. 9 depicts examples of asymmetric adaptors.

FIG. 10 depicts asympetric adaptors.

FIGS. 11A-11D depict the process of generating asymmetric sequencing libraries.

FIG. 12A depicts the structure of a molecule in an asymmetric library.

FIG. 12B depicts the amplification of an asymmetric library.

FIG. 13 depicts paired end sequencing of the first strand from adapter A.

FIG. 14 depicts paired end sequencing of the second strand from adapter B.

FIG. 15 depicts sequencing of the first and second strands.

FIG. 16 depicts an overlap of the location of sequencing reads from the first strand and second strand.

FIG. 17 depicts base calls from the first and second strand.

### DETAILED DESCRIPTION

Provided herein are methods and compositions for paired end sequencing without turnaround chemistry. The methods and compositions described herein provide benefits over other sequencing methods. The methods described herein allow for error correction on the sequencing by sequencing both the Watson and Crick strands. The methods described herein also eliminate the need for unique molecular identifiers (UMIs) and randomly sheared DNA while increasing the effective number of reads per flow cell as PCT-free methods can be used for high-quality base calls. The methods further eliminate the need for circularization during library preparation of concatamers (CATs).

### I. METHODS

### A. Library Preparation

The methods described herein may involve preparation of a library for sequencing. In certain aspects, the methods describe ligating two separate adaptors to a double-stranded linear nucleic acid molecule to produce a single-stranded circular nucleic acid molecule. In certain aspects, the single-stranded circular nucleic acid molecule is amplified to create a single-stranded concatemer containing both the adaptor sequences and both the forward and reverse sequences of the double-stranded linear nucleic acid molecule.

In some embodiments, a double-stranded sample nucleic acid is prepared for sequencing by ligating a first adaptor described herein and a second adaptor described herein to the double-stranded nucleic acid. In some embodiments, two adapters are ligated to a double-stranded nucleic acid molecule to produce a single stranded circular nucleic acid molecule. In some embodiments, the single-stranded circular nucleic acid molecule is amplified using rolling circle amplification. In some embodiments, the single-stranded circular nucleic acid molecule is used as a template to create a concatemer for use in sequence. In some embodiments, the single-stranded circular nucleic acid molecule is used as a template to create a single-stranded nucleic acid molecule comprising copies of the first strand of the double-stranded nucleic acid molecule and copies of the second strand of the double-stranded nucleic acid molecule.

The methods described used adaptors described herein. The adaptors may comprise loops. The adaptors may comprise hairpins. The adaptors may comprise stem loops. The adaptors may comprise primer hybridization sites. The first adaptor may comprise a first primer hybridization site and the second adaptor may comprise a second primer hybridization site. The first adaptor and the second adaptor may comprise different sequences.

### 1. Creating the Single-Stranded Circular Template

In some embodiments, adaptors are ligated to a linear double-stranded nucleic acid molecule to produce a single-stranded circular nucleic acid molecule.

### a. Standard Dual Sequencing

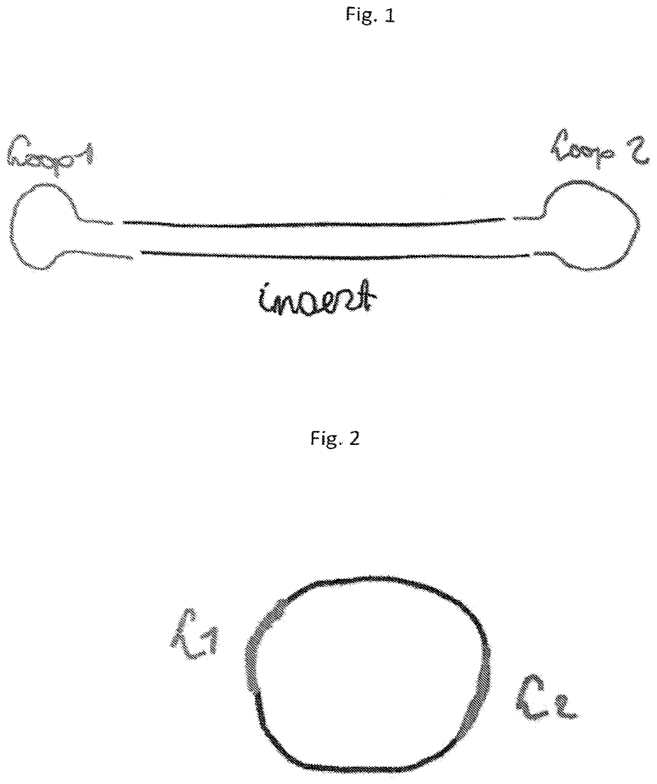

In some embodiments, an adaptor is ligated to a first end of a double-stranded nucleic acid molecule. In some embodiments, a second adaptor is ligated to a second end of a double-stranded nucleic acid molecule. In some embodiments, a first adaptor comprising a hairpin and a second adaptor comprising a hairpin are ligated to a double stranded linear nucleic acid molecule, as depicted in FIG. 1. Ligating the two adaptors to the double-stranded linear nucleic acid molecules may produce a single-stranded circular nucleic acid molecule, such as depicted in FIG. 2. The single-stranded circular nucleic acid molecule may comprise both the sequence of the first adaptor, the second adaptor, the forward strand of the double-stranded linear nucleic acid molecule and the reverse strand of the double-stranded linear nucleic acid molecule.

The first adaptor may be ligated to the first end of the double-stranded nucleic acid molecule before the second adaptor is ligated to the second end of the double-stranded nucleic acid molecule.

Alternatively, the nucleic acid may be immobilized on a solid support. The solid support may be a bead. A first adaptor may be ligated. The nucleic acid may then be cut or eluted from the bead and the second adaptor may be ligated.

Alternatively, two single ligation steps with two different adaptors may occur, followed by enrichment of inserts with two different adaptors. The two different adaptors may be combined at a 1:1 ratio. Ligation may occur simultaneously.

Alternatively, a Y-shaped adaptor may be ligated. Following ligation of the Y-shaped adaptors to each end of the nucleic acid, a round of PCR amplification will produce a nucleic acid sequence with a different adaptor on each end.

In some embodiments, the method comprises: providing a double-stranded nucleic acid molecule comprising a first strand and a second strand hybridized to the first strand; ligating adaptors to ends of the double-stranded nucleic acid molecule to yield a circular nucleic acid molecule; and using the circular nucleic acid molecule as a template to generate a single-stranded nucleic acid molecule comprising copies of the first strand and said second strand. In some embodiments, the first adaptor and the second adaptor are different.

### b. Asymmetric Dual Sequencing

In some embodiments, the methods comprise preparing two-single stranded circular molecules from one double-stranded linear molecule.

The sample nucleic acid may be contacted with one or more first adaptors to form a sample-adaptor complex. The one or more first adaptors may be an adaptor described herein. The one or more first adaptors may prevent ligation between the 5′ end of the forward sample strand and the 5′ end of the reverse sample strand. The adaptor may block ligation via a gap, flap, or a blocking group as described herein.

In some embodiments, the sample-adaptor complex is contacted with a polymerase to form a forward double-stranded sample-adaptor complex comprising the forward strand, the first adaptor, and a sequence complementary to the forward strand. In some embodiments, the sample-adaptor complex is contacted with a polymerase to form a reverse double-stranded sample-adaptor complex comprising the reverse sample strand, the first adaptor and a sequence complementary to the reverse sample strand. In some embodiments, the polymerase uses the forward strand of the sample to extend the 5′ end of the adaptor. In some embodiments, the polymerase uses the reverse strand of the sample to extend the 5′ end of the adaptor. In some embodiments, both a forward double-stranded sample-adaptor complex and a reverse double-stranded sample adaptor complex is created.

In some embodiments, the forward double-stranded sample-adaptor complex comprises a double-stranded DNA sequence comprising one blunt end and one looped end. The blunt end may comprise a 5′ end and a 3′ end of the sample nucleic acid. The looped end may comprise the first adaptor.

In some embodiments, the reverse double-stranded sample-adaptor complex comprises a double-stranded DNA sequence comprising one blunt end and one looped end. The blunt end may comprise a 5′ end and a 3′ end of the sample nucleic acid. The looped end may comprise the first adaptor.

In some embodiments, a second adaptor is ligated to the forward double-stranded adaptor complex. In some embodiments, the second adaptor and the forward double-stranded adaptor complex are ligated to form a single-stranded circular DNA molecule comprising the first adaptor, the forward strand of the sample DNA, the second adaptor, and a sequence complementary to the forward strand of the sample DNA.

In some embodiments, a second adaptor is ligated to the reverse double-stranded adaptor complex. In some embodiments, the second adaptor and the reverse double-stranded adaptor complex are ligated to form a single-stranded circular DNA molecule comprising the first adaptor, the reverse strand of the sample DNA, the second adaptor, and a sequence complementary to the reverse strand of the sample DNA.

The second adaptor may comprise a second primer hybridization site. The second adaptor may comprise a hairpin. The second adaptor may be different than the first adaptor. The second adaptor may comprise a different primer hybridization site than the first adaptor. The second adaptor may comprise a 5′ end that is available for ligation. The second adaptor may comprise a 3′ end that is available for ligation.

In some embodiments, the methods comprise contacting the sample nucleic acid with one or more first adaptors to form a sample-adaptor complex, wherein the one or more first adaptors prevent ligation between the 5′ end of the forward sample strand and the 5′ end of the reverse sample strand; extending with a polymerase to form a forward double-stranded sample-adaptor complex comprising the forward strand, the first adaptor, and a sequence complementary to the forward strand and a reverse double-stranded sample-adaptor complex comprising the reverse sample strand, the first adaptor and a sequence complementary to the reverse sample strand; and ligating one or more second adaptors to the forward double-stranded sample-adaptor complex and the reverse double-stranded sample-adaptor complex to form a circular forward-adaptor complex and a circular reverse-adaptor complex.

### 2. Generating Polynucleotide Concatemers by Rolling Circle Replication

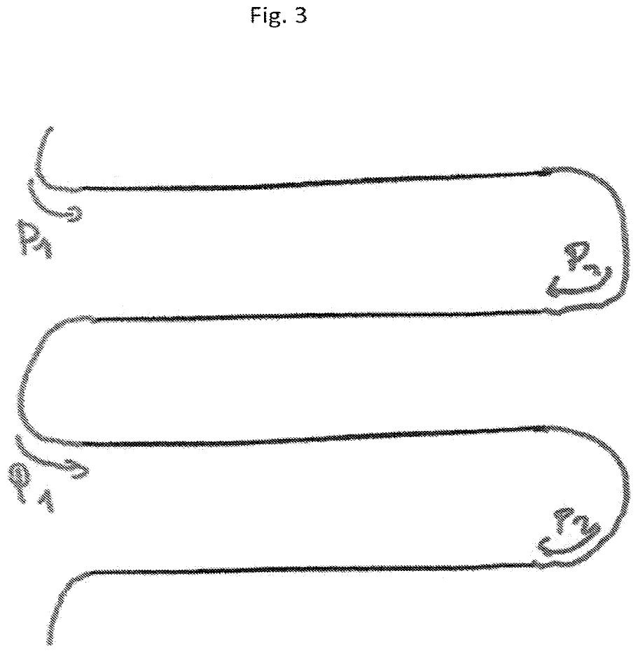

In certain aspect, the methods described herein comprise producing a concatemer from the single-stranded circular nucleic acids produced by the methods described herein. In some embodiments, rolling circle replication is used to produced the concatemer. In some embodiments, the concatemer is a single-stranded nucleic acid molecule comprising copies of the first strand and the second strand of the nucleic acid molecule. A non-limiting example of the concatemer can be depicted in FIG. 3.

In one aspect of the invention, single molecules comprise concatemers of polynucleotides, usually polynucleotide analytes, i.e. target sequences, that have been produce in a conventional rolling circle replication (RCR) reaction. Guidance for selecting conditions and reagents for RCR reactions is available in many references available to those of ordinary skill, as evidence by the following that are incorporated by reference: Kool, U.S. Pat. No. 5,426,180; Lizardi, U.S. Pat. Nos. 5,854,033 and 6,143,495; Landegren, U.S. Pat. No. 5,871,921; and the like. Generally, RCR reaction components comprise single stranded DNA circles, one or more primers that anneal to DNA circles, a DNA polymerase having strand displacement activity to extend the 3′ ends of primers annealed to DNA circles, nucleoside triphosphates, and a conventional polymerase reaction buffer. Such components are combined under conditions that permit primers to anneal to DNA circles and be extended by the DNA polymerase to form concatemers of DNA circle complements. An exemplary RCR reaction protocol is as follows: In a 50 μL reaction mixture, the following ingredients are assembled: 2-50 pmol circular DNA, 0.5 units/μL phage φ29 DNA polymerase, 0.2 μg/μL BSA, 3 mM dNTP, 1×φ29 DNA polymerase reaction buffer (Amersham). The RCR reaction is carried out at 30 °C for 12 hours. In some embodiments, the concentration of circular DNA in the polymerase reaction may be selected to be low (approximately 10-100 billion circles per ml, or 10-100 circles per picoliter) to avoid entanglement and other intermolecular interactions.

Preferably, concatemers produced by RCR are approximately uniform in size; accordingly, in some embodiments, methods of making arrays of the invention may include a step of size-selecting concatemers. For example, in one aspect, concatemers are selected that as a population have a coefficient of variation in molecular weight of less than about 30%; and in another embodiment, less than about 20%. In one aspect, size uniformity is further improved by adding low concentrations of chain terminators, such ddNTPs, to the RCR reaction mixture to reduce the presence of very large concatemers, e.g. produced by DNA circles that are synthesized at a higher rate by polymerases. In one embodiment, concentrations of ddNTPs are used that result in an expected concatemer size in the range of from 50-250 Kb, or in the range of from 50-100 Kb. In another aspect, concatemers may be enriched for a particular size range using a conventional separation techniques, e.g. size-exclusion chromatography, membrane filtration, or the like.

### 3. Synthetic Nucleic Acid Probes

Probe sequences of random arrays may be derived from virtually any population of nucleic acid fragments that can produce useful information in a hybridization assay. In one aspect, probe sequences of random arrays are extracted or derived from nucleic acids in a sample. Exemplary samples include, but are not limited to, samples from a population of individuals or organisms, a single patient, a single tissue from multiple patients, multiple tissues from one or more patients, an organism of economic interest, a community of microorganisms, a collection of synthetic nucleic acids (e.g. the set of all nucleic acid sequences having a length selected from the range of from 10-20), or the like. In another aspect, probe sequences may be derived from a genomic DNA library, cDNA library, cRNA library, siRNA library, or other classes of natural nucleic acids. In another aspect, the invention provides random arrays for comparing gene expression or copy number abundances among different biological samples; in such embodiment, probe sequences may be derived from a consensus or reference library of DNA fragments. Typically, the nucleotide sequences from a reference library are known and the sequences typically are listed in sequence databases, such as Genbank, Embl, or the like. In one aspect, a reference library of DNA may comprise a cDNA library or genomic library from a known cell type or tissue source. For example, a reference library of DNA may comprise a cDNA library or a genomic library derived from the tissue of a healthy individual and a test library of DNA (from which target sequences are derived) may comprise a cDNA library or genomic library derived from the same tissue of a diseased individual. Reference libraries of DNA may also comprise an assembled collection of individual polynucleotides, cDNAs, genes, or exons thereof, e.g. genes or exons encoding all or a subset of known p53 variants, genes of a signal transduction pathway, or the like. The DNA use for making probes may be enriched through various procedures. For example, variable regions between 2 and 20 or between 20 and 2000 individuals may be collected using mismatch cutting enzymes or other procedures to make arrays enriched for polymorphisms.

In one aspect, probe sequences are synthetic polynucleotides having predetermined sequences. In one embodiment, synthetic probe sequences are selected for detecting protein-DNA binding, e.g. Gronostajski, Nucleic Acids Research, 15: 5545-5559 (1987); Oliphant et al, Gene, 44: 177-183 (1986); Oliphant et al, Meth. Enzymol., 155: 568-582 (1987); which references are incorporated by reference. In one aspect, probe sequences for such use may have the following form: “oligo1-NNN . . . NNN-oligo2”, where “oligo1” and “oligo2” are oligonucleotides of known sequence, e.g. primer binding sites, which sandwich a random sequence region “NNN . . . NNN”, which may vary in length and composition. In one form, the random sequence region has a length in the range of from 6 to 20, or in the range of from 8 to 16. In another form, “N” is any of the four natural nucleotides. In another aspect, preparation of selected synthetic probes (for example, between about 20 to 100 bases in length) may be produced individually or in various pools. One pool example is 10-10,000 probes of different sequences mixed and extended with the same 5-15 base sequence in the same synthesis. These probes may be tagged for decoding or decoded directly by sequencing a portion of, or the entire, probe. 4-15 bases is sufficient for identifying thousands to millions of sequences.

Genomic DNA is obtained using conventional techniques, for example, as disclosed in Sambrook et al., supra, 1999; Current Protocols in Molecular Biology, Ausubel et al., eds. (John Wiley and Sons, Inc., NY, 1999), or the like, Important factors for isolating genomic DNA include the following: 1) the DNA is free of DNA processing enzymes and contaminating salts; 2) the entire genome is equally represented; and 3) the DNA fragments are between about 5,000 and 100,000 bp in length. In many cases, no digestion of the extracted DNA is required because shear forces created during lysis and extraction will generate fragments in the desired range. In another embodiment, shorter fragments (1-5 kb) can be generated by enzymatic fragmentation using restriction endonucleases. In one embodiment, 10-100 genome-equivalents of DNA ensure that the population of fragments covers the entire genome. In some cases, it is advantageous to provide carrier DNA, e.g. unrelated circular synthetic double-stranded DNA, to be mixed and used with the sample DNA whenever only small amounts of sample DNA are available and there is danger of losses through nonspecific binding, e.g. to container walls and the like.

In generating fragments in either stage, fragments may be derived from either an entire genome or it may be derived from a selected subset of a genome. Many techniques are available for isolating or enriching fragments from a subset of a genome, as exemplified by the following references that are incorporated by reference: Kandpal et al (1990), Nucleic Acids Research, 18: 1789-1795; Callow et al, U.S. patent publication 2005/0019776; Zabeau et al, U.S. Pat. No. 6,045,994; Deugau et al, U.S. Pat. No. 5,508,169; Sibson, U.S. Pat. No. 5,728,524; Guilfoyle et al, U.S. Pat. No. 5,994,068; Jones et al, U.S. patent publication 2005/0142577; Gullberg et al, U.S. patent publication 2005/0037356; Matsuzaki et al, U.S. patent publication 2004/0067493; and the like.

For mammalian-sized genomes, an initial fragmentation of genomic DNA can be achieved by digestion with one or more “rare” cutting restriction endonucleases, such as Not I, Asc I, Bae I, CspC I, Pac I, Fse I, Sap I, Sfi I, Psr I, or the like. The resulting fragments can be used directly, or for genomes that have been sequenced, specific fragments may be isolated from such digested DNA for subsequent processing. Genomic DNA is digested with a rare cutting restriction endonuclease to generate fragments, after which the fragments are further digested for a short period (i.e. the reaction is not allowed to run to completion) with a 5′ single stranded exonuclease, such as λ exonuclease, to expose sequences adjacent to restriction site sequences at the end of the fragments. Such exposed sequences will be unique for each fragment.

### B. Clonal Amplification

In some embodiments, the methods comprise clonally amplifying a single-stranded concatemer described herein. In some embodiments, the single-stranded DNA molecule is produced by contacting a sample double-stranded DNA molecule comprising a template sequence with a first adaptor comprising a first adaptor sequence and a second adaptor comprising a second adaptor sequence to produce a circular DNA molecule, wherein the first adaptor sequence comprises a first primer hybridization sequence and wherein the second adaptor sequence comprises a second primer hybridization sequence; and contacting the circular DNA molecule with a polymerizing enzyme to produce a single-stranded DNA molecule via rolling circle amplification, wherein the single-stranded DNA molecule comprises a sequence comprising at least the first adaptor sequence, the template sequence, the second adaptor sequence, and a sequence complementary to the template sequence.

The methods may involve hybridizing a first primer to the first primer hybridization sequence site on the first adaptor sequence in the single-stranded DNA molecule or concatamer. In some embodiments, a polymerase is used to extend the first primer to produce a nascent sequence complementary to the template sequence. In some embodiments, the methods involve hybridizing a plurality of first primers to a plurality of first primer hybridization sequence sites on a plurality of first adaptor sequences in the single-stranded DNA molecule or concatemer.

In some embodiments, the first adaptor sequence comprises a first blocking sequence. In some embodiments, the second adaptor sequence comprises a second blocking sequence. In some embodiments, the first blocking sequence is different than the second blocking sequence. In some embodiments, the first blocking sequence is the same as the first primer hybridization sequence. In some embodiments, the second blocking sequence is the same as the second primer hybridization sequence.

In some embodiments, the methods comprise contacting the single stranded DNA molecule with a blocking molecule that binds to the blocking sequence. The blocking molecule may prevent extension of a nascent sequence beyond the first adaptor.

The blocking molecule may comprise an oligonucleotide. The oligonucleotide may comprise a locked nucleic acid, a psoralen modified nucleic acid, a MGB modified nucleic acid or G-quadruplex oligo. The blocking molecule may comprise a peptide or DNA binding protein. The peptide or protein may bind to the blocking sequence on the adaptor. The DNA binding protein may be a Cas protein. The DNA binding protein may be a Tus protein.

Extending from the first primer site may result in a DNA molecule comprising: a first DNA strand comprising: a first segment comprising a first sequence; wherein the first segment is ligated to a first hairpin segment comprising a first hairpin sequence at the 3′ end of the first hairpin segment; a second segment comprising a second sequence that is complementary to the first sequence; wherein the second segment is ligated to the first hairpin segment at the 5′ end of the second segment and wherein the second segment is ligated to a second hairpin segment comprising a second hairpin sequence at the 3′ end of the second segment; a third segment comprising the first sequence, wherein the third segment is ligated to the second hairpin sequence at the 5′ end of the third segment and wherein the third segment is ligated to a third hairpin segment comprising the first hairpin sequence at the 3′ end; a fourth segment comprising the second sequence; wherein the fourth segment is ligated to the third hairpin segment at the 5′ end of the fourth segment; and a second DNA strand comprising a sequence complementary to the first sequence.

In some embodiments, once the nascent sequences are extended to the blocking sequence, the concatemer is contacted with a second primer. The primer may hybridize to a second primer hybridization site sequence in the second adaptor sequence. The reverse template strand may be extended using a polymerase.

### 4. Solid Phase Surfaces for Constructing Random Arrays

A wide variety of supports may be used with the invention. In one aspect, supports are rigid solids that have a surface, preferably a substantially planar surface so that single molecules to be interrogated are in the same plane. The latter feature permits efficient signal collection by detection optics, for example. In another aspect, solid supports of the invention are nonporous, particularly when random arrays of single molecules are analyzed by hybridization reactions requiring small volumes. Suitable solid support materials include materials such as glass, polyacrylamide-coated glass, ceramics, silica, silicon, quartz, various plastics, and the like. In one aspect, the area of a planar surface may be in the range of from 0.5 to 4 cm². In one aspect, the solid support is glass or quartz, such as a microscope slide, having a surface that is uniformly silanized. This may be accomplished using conventional protocols, e.g. acid treatment followed by immersion in a solution of 3-glycidoxypropyl trimethoxysilane, N,N-diisopropylethylamine, and anhydrous xylene (8:1:24 v/v) at 80 °C, which forms an epoxysilanized surface. e.g. Beattie et a (1995), Molecular Biotechnology, 4: 213. Such a surface is readily treated to permit end-attachment of capture oligonucleotides, e.g. by providing capture oligonucleotides with a 3′ or 5′ triethylene glycol phosphoryl spacer (see Beattie et al, cited above) prior to application to the surface. Many other protocols may be used for adding reactive functionalites to glass and other surfaces, as evidenced by the disclosure in Beaucage (cited above).

Whenever enzymatic processing is not required, capture oligonucleotides may comprise non-natural nucleosidic units and/or linkages that confer favorable properties, such as increased duplex stability; such compounds include, but not limited to, peptide nucleic acids (PNAs), locked nucleic acids (LNA), oligonucleotide N3′→P5′ phosphoramidates, oligo-2′-O-alkylribonucleotides, and the like.

In embodiments of the invention in which patterns of discrete spaced apart regions are required, photolithography, electron beam lithography, nano imprint lithography, and nano printing may be used to generate such patterns on a wide variety of surfaces, e.g. Pirrung et al, U.S. Pat. No. 5,143,854; Fodor et al, U.S. Pat. No. 5,774,305; Guo, (2004) Journal of Physics D: Applied Physics, 37: R123-141; which are incorporated herein by reference.

In one aspect, surfaces containing a plurality of discrete spaced apart regions are fabricated by photolithography. A commercially available, optically flat, quartz substrate is spin coated with a 100-500 nm thick layer of photo-resist. The photo-resist is then baked on to the quartz substrate. An image of a reticle with a pattern of regions to be activated is projected onto the surface of the photo-resist, using a stepper. After exposure, the photo-resist is developed, removing the areas of the projected pattern which were exposed to the UV source. This is accomplished by plasma etching, a dry developing technique capable of producing very fine detail. The substrate is then baked to strengthen the remaining photo-resist. After baking, the quartz wafer is ready for functionalization. The wafer is then subjected to vapor-deposition of 3-aminopropyldimethylethoxysilane. The density of the amino functionalized monomer can be tightly controlled by varying the concentration of the monomer and the time of exposure of the substrate. Only areas of quartz exposed by the plasma etching process may react with and capture the monomer. The substrate is then baked again to cure the monolayer of amino-functionalized monomer to the exposed quartz. After baking, the remaining photo-resist may be removed using acetone. Because of the difference in attachment chemistry between the resist and silane, aminosilane-functionalized areas on the substrate may remain intact through the acetone rinse. These areas can be further functionalized by reacting them with p-phenylenediisothiocyanate in a solution of pyridine and N-N-dimethlyformamide. The substrate is then capable of reacting with amine-modified oligonucleotides. Alternatively, oligonucleotides can be prepared with a 5′-carboxy-modifier-c10 linker (Glen Research). This technique allows the oligonucleotide to be attached directly to the amine modified support, thereby avoiding additional functionalization steps.

In another aspect, surfaces containing a plurality of discrete spaced apart regions are fabricated by nano-imprint lithography (NIL). For DNA array production, a quartz substrate is spin coated with a layer of resist, commonly called the transfer layer. A second type of resist is then applied over the transfer layer, commonly called the imprint layer. The master imprint tool then makes an impression on the imprint layer. The overall thickness of the imprint layer is then reduced by plasma etching until the low areas of the imprint reach the transfer layer. Because the transfer layer is harder to remove than the imprint layer, it remains largely untouched. The imprint and transfer layers are then hardened by heating. The substrate is then put into a plasma etcher until the low areas of the imprint reach the quartz. The substrate is then derivatized by vapor deposition as described above.

In another aspect, surfaces containing a plurality of discrete spaced apart regions are fabricated by nano printing. This process uses photo, imprint, or e-beam lithography to create a master mold, which is a negative image of the features required on the print head. Print heads are usually made of a soft, flexible polymer such as polydimethylsiloxane (PDMS). This material, or layers of materials having different properties, are spin coated onto a quartz substrate. The mold is then used to emboss the features onto the top layer of resist material under controlled temperature and pressure conditions. The print head is then subjected to a plasma based etching process to improve the aspect ratio of the print head, and eliminate distortion of the print head due to relaxation over time of the embossed material. Random array substrates are manufactured using nano-printing by depositing a pattern of amine modified oligonucleotides onto a homogenously derivatized surface. These oligo-nucleotides would serve as capture probes for the RCR products. One potential advantage to nano-printing is the ability to print interleaved patterns of different capture probes onto the random array support. This would be accomplished by successive printing with multiple print heads, each head having a differing pattern, and all patterns fitting together to form the final structured support pattern. Such methods allow for some positional encoding of DNA elements within the random array. For example, control concatemers containing a specific sequence can be bound at regular intervals throughout a random array.

In still another aspect, a high density array of capture oligonucleotide spots of sub micron size is prepared using a printing head or imprint-master prepared from a bundle, or bundle of bundles, of about 10,000 to 100 million optical fibers with a core and cladding material. By pulling and fusing fibers a unique material is produced that has about 50-1000 nm cores separated by a similar or 2-5 fold smaller or larger size cladding material. By differential etching (dissolving) of cladding material a nano-printing head is obtained having a very large number of nano-sized posts. This printing head may be used for depositing oligonucleotides or other biological (proteins, oligopeptides, DNA, aptamers) or chemical compounds such as silane with various active groups. In one embodiment the glass fiber tool is used as a patterned support to deposit oligonucleotides or other biological or chemical compounds. In this case only posts created by etching may be contacted with material to be deposited. Also, a flat cut of the fused fiber bundle may be used to guide light through cores and allow light-induced chemistry to occur only at the tip surface of the cores, thus eliminating the need for etching. In both cases, the same support may then be used as a light guiding/collection device for imaging fluorescence labels used to tag oligonucleotides or other reactants. This device provides a large field of view with a large numerical aperture (potentially >1). Stamping or printing tools that perform active material or oligonucleotide deposition may be used to print 2 to 100 different oligonucleotides in an interleaved pattern. This process requires precise positioning of the print head to about 50-500 nm. This type of oligonucleotide array may be used for attaching 2 to 100 different DNA populations such as different source DNA. They also may be used for parallel reading from sub-light resolution spots by using DNA specific anchors or tags. Information can be accessed by DNA specific tags, e.g. 16 specific anchors for 16 DNAs and read 2 bases by a combination of 5-6 colors and using 16 ligation cycles or one ligation cycle and 16 decoding cycles. This way of making arrays is efficient if limited information (e.g. a small number of cycles) is required per fragment, thus providing more information per cycle or more cycles per surface.

In one embodiment “inert” concatemers are used to prepare a surface for attachment of test concatemers. The surface is first covered by capture oligonucleotides complementary to the binding site present on two types of synthetic concatemers; one is a capture concatemer, the other is a spacer concatemer. The spacer concatemers do not have DNA segments complementary to the adapter used in preparation of test concatemers and they are used in about 5-50, preferably 10× excess to capture concatemers. The surface with capture oligonucleotide is “saturated” with a mix of synthetic concatemers (prepared by chain ligation or by RCR) in which the spacer concatemers are used in about 10-fold (or 5 to 50-fold) excess to capture concatemers. Because of the ~10:1 ratio between spacer and capture concatemers, the capture concatemers are mostly individual islands in a sea of spacer concatemers. The 10:1 ratio provides that two capture concatemers are on average separated by two spacer concatemers. If concatemers are about 200 nm in diameter, then two capture concatemers are at about 600 nm center-to-center spacing. This surface is then used to attach test concatemers or other molecular structures that have a binding site complementary to a region of the capture concatemers but not present on the spacer concatemers. Capture concatemers may be prepared to have less copies than the number of binding sites in test concatemers to assure single test concatemer attachment per capture concatemer spot. Because the test DNA can bind only to capture concatemers, an array of test concatemers may be prepared that have high site occupancy without congregation. Due to random attachment, some areas on the surface may not have any concatemers attached, but these areas with free capture oligonucleotide may not be able to bind test concatemers since they are designed not to have binding sites for the capture oligonucleotide. An array of individual test concatemers as described would not be arranged in a grid pattern. An ordered grid pattern should simplify data collection because less pixels are needed and less sophisticated image analysis systems are needed also.

In one aspect, multiple arrays of the invention may be place on a single surface. For example, patterned array substrates may be produced to match the standard 96 or 384 well plate format. A production format can be an 8×12 pattern of 6 mm×6 mm arrays at 9 mm pitch or 16×24 of 3.33 mm×3.33 mm array at 4.5 mm pitch, on a single piece of glass or plastic and other optically compatible material. In one example each 6 mm×6 mm array consists of 36 million 250-500 nm square regions at 1 micrometer pitch. Hydrophobic or other surface or physical barriers may be used to prevent mixing different reactions between unit arrays.

### 5. Detection

As described herein, each of the detection methods and systems required cycled detection to achieve sub-diffraction limited imaging. Cycled detection includes the binding and imaging or probes, such as antibodies or nucleotides, bound to detectable labels that are capable of emitting a visible light optical signal. By using positional information from a series of images of a field from different cycles, deconvolution to resolve signals from densely packed substrates can be used effectively to identify individual optical signals from signals obscured due to the diffraction limit of optical imaging. After multiple cycles the precise location of the molecule will become increasingly more accurate. Using this information additional calculations can be performed to aid in crosstalk correction regarding known asymmetries in the crosstalk matrix occurring due to pixel discretization effects.

Methods and systems using cycled probe binding and optical detection are described in US Publication No. 2015/0330974, Digital Analysis of Molecular Analytes Using Single Molecule Detection, published Nov. 19, 2015, incorporated herein by reference in its entirety.

In some embodiments, the raw images are obtained using sampling that is at least at the Nyquist limit to facilitate more accurate determination of the oversampled image. Increasing the number of pixels used to represent the image by sampling in excess of the Nyquist limit (oversampling) increases the pixel data available for image processing and display.

Theoretically, a bandwidth-limited signal can be perfectly reconstructed if sampled at the Nyquist rate or above it. The Nyquist rate is defined as twice the highest frequency component in the signal. Oversampling improves resolution, reduces noise and helps avoid aliasing and phase distortion by relaxing anti-aliasing filter performance requirements. A signal is said to be oversampled by a factor of N if it is sampled at N times the Nyquist rate.

Thus, in some embodiments, each image is taken with a pixel size no more than half the wavelength of light being observed. In some embodiments, a pixel size of 162.5 nm×162.5 nm is used in detection to achieve sampling at or above the Nyquist limit. Sampling at a frequency of at least the Nyquist limit during raw imaging of the substrate is preferred to optimize the resolution of the system or methods described herein. This can be done in conjunction with the deconvolution methods and optical systems described herein to resolve features on a substrate below the diffraction limit with high accuracy.

### c. Processing Images from Different Cycles

There are several barriers overcome by the present invention to achieve sub-diffraction limited imaging.

Pixelation error is present in raw images and prevents identification of information present from the optical signals due to pixelation. Sampling at least at the Nyquist frequency and generation of an oversampled image as described herein each assist in overcoming pixilation error.

The point-spread (PSF) of various molecules overlap because the PSF size is greater than the pixel size (below Nyquist) and because the center-to-center spacing is so small that crosstalk due to spatial overlap occurs. Nearest neighbor variable regression (for center-to center crosstalk) can be used to help with deconvolution of multiple overlapping optical signals. But this can be improved if we know the relative location of each analyte on the substrate and have good alignment of images of a field.

After multiple cycles the precise location of the molecule will become increasingly more accurate. Using this information additional calculations can be performed to aid in deconvolution by correcting for known asymmetries in the spatial overlap of optical signals occurring due to pixel discretization effects and the diffraction limit. They can also be used to correct for overlap in emission spectrum from different emission spectrum.

Highly accurate relative positional information for each analyte can be achieved by overlaying images of the same field from different cycles to generate a distribution of measured peaks from optical signals of different probes bound to each analyte. This distribution can then be used to generate a peak signal that corresponds to a single relative location of the analyte. Images from a subset of cycles can be used to generate relative location information for each analyte. In some embodiments, this relative position information is provided in a localization file.

The specific area imaged for a field for each cycle may vary from cycle to cycle. Thus, to improve the accuracy of identification of analyte position for each image, an alignment between images of a field across multiple cycles can be performed. From this alignment, offset information compared to a reference file can then be identified and incorporated into the deconvolution algorithms to further increase the accuracy of deconvolution and signal identification for optical signals obscured due to the diffraction limit. In some embodiments, this information is provided in a Field Alignment File.

### d. Signal Detection (Cross-Talk/Nearest Neighbor)

Once relative positional information is accurately determined for analytes on a substrate and field images from each cycle are aligned with this positional information, analysis of each oversampled image using crosstalk and nearest neighbor regression can be used to accurately identify an optical signal from each analyte in each image.

In some embodiments, a plurality of optical signals obscured by the diffraction limit of the optical system are identified for each of a plurality of biomolecules immobilized on a substrate and bound to probes comprising a detectable label. In some embodiments, the probes are incorporated nucleotides and the series of cycles is used to determine a sequence of a polynucleotide immobilized on the array using single molecule sequencing by synthesis.

### e. Simulations of Deconvolution Applied to Images

Molecular densities are limited by crosstalk from neighboring molecules. Acceptable crosstalk levels at or below 25% with 2× oversample occurs for pitches at or above 275 nm. Acceptable crosstalk levels at or below 25% with 4× deconvolution using the point spread function of the optical system occurs for pitches at or above 210 nm.

The physical size of the molecule will broaden the spot roughly half the size of the binding area. For example, for an 80 nm spot the pitch will be increased by roughly 40 nm. Smaller spot sizes may be used, but this will have the trade-off that fewer copies will be allowed and greater illumination intensity will be required. A single copy provides the simplest sample preparation but requires the greatest illumination intensity.

Methods for sub-diffraction limit imaging discussed to this point involve image processing techniques of oversampling, deconvolution and crosstalk correction. Described herein are methods and systems that incorporate determination of the precise relative location analytes on the substrate using information from multiple cycles of probe optical signal imaging for the analytes. Using this information additional calculations can be performed to aid in crosstalk correction regarding known asymmetries in the crosstalk matrix occurring due to pixel discretization effects.

### C. Methylation

In some embodiments, the methods comprise detecting a methylation signature. In some embodiments, a methyltransferase is used during sequence amplification to maintain the DNA methylation status. The methyltransferase may include a DNA methyltransferase (DNMT). Bisulfite conversion of DNA results in conversion of unmodified cytosine (C) to uracil (U) that will be read as thymine (T) upon sequencing of PCR amplified DNA. Both 5meC and 5hmC are protected against conversion and will not be converted to U. Therefore they will both be read as C upon sequencing. In some embodiments, bisulfite conversion occurs after generation of the concatemer. In some embodiments, bisulfite conversion occurs after sequencing the concatemer. In some embodiments, an additional round of sequencing is performed after bisulfite conversion.

### II. COMPOSITIONS OF MATTER

### D. Adaptor Molecules

The methods and compositions described herein comprise at least one adaptor molecule. In some embodiments, the methods comprise a plurality of adaptor molecules. In some embodiments, the methods comprise a first adaptor molecule and a second adaptor molecule, wherein the first and second adaptor molecule is different. In some embodiments, the methods comprise an asymmetric adaptor molecule. In some embodiments, the adaptor comprises a hairpin. In some embodiments, the adaptor comprises a blocking group. In some embodiments, the adaptor comprises at least a first primer hybridization site.

### 6. Asymmetric Adaptor Molecules

In certain aspects, disclosed herein is an adaptor, comprising: a first nucleic acid strand comprising a 5′ segment and a 3′ segment, wherein the 3′ segment of the first nucleic acid strand comprises a hairpin and the 5′ segment of the first nucleic acid strand comprises an overhang; and a second nucleic acid strand comprising a 3′ segment and a 5′ segment, wherein the 3′ segment of the second nucleic acid strand comprises a sequence complementary to the 5′ overhang of the first nucleic acid strand and wherein the 5′ segment of the second nucleic acid strand comprises a nucleic acid sequence that is not complementary to the first nucleic acid molecule, and wherein the 5′ segment of the second nucleic acid strand comprises a blocking group.

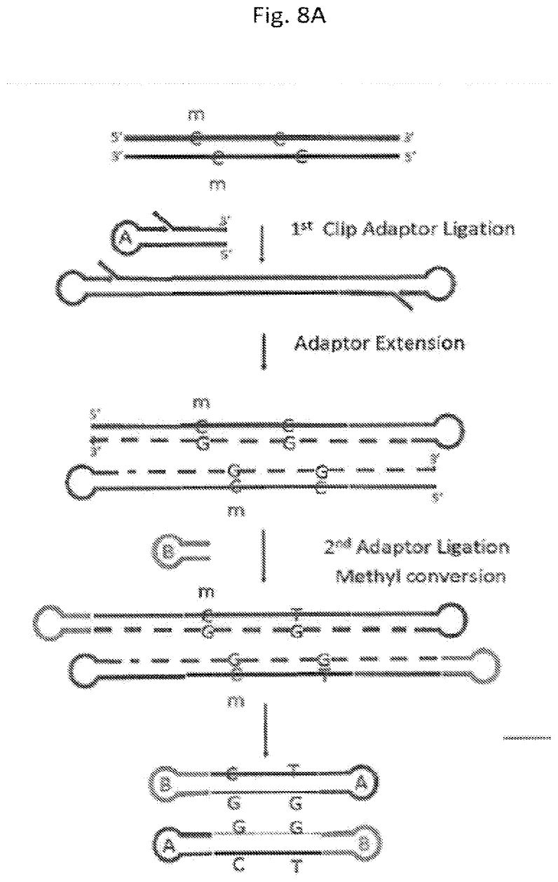

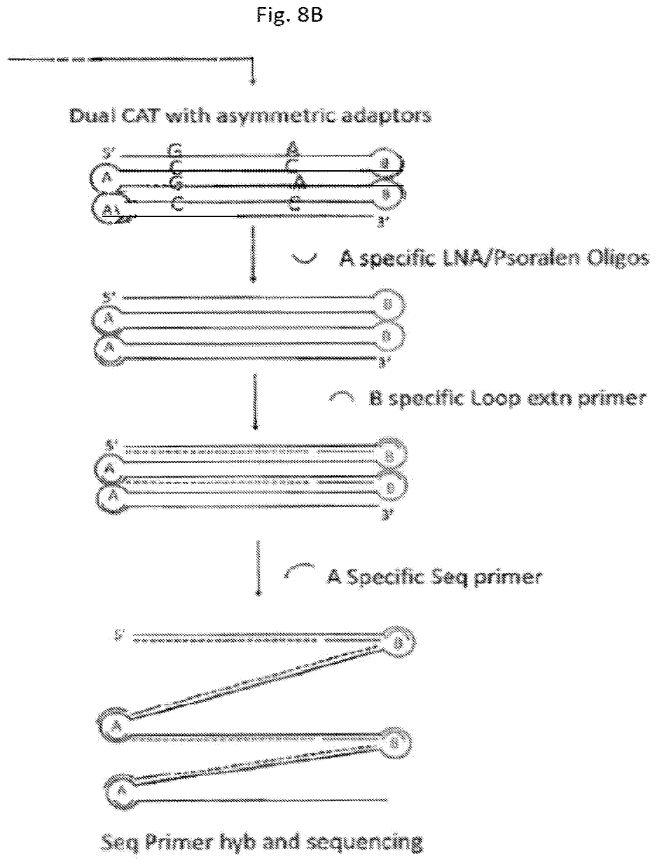

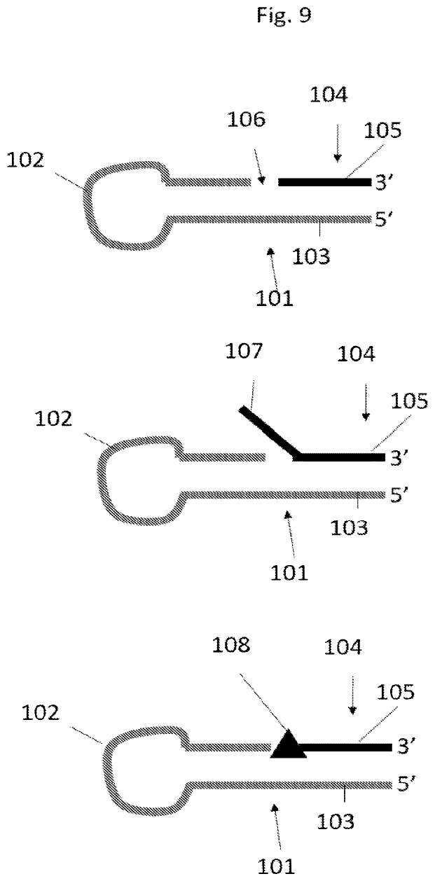

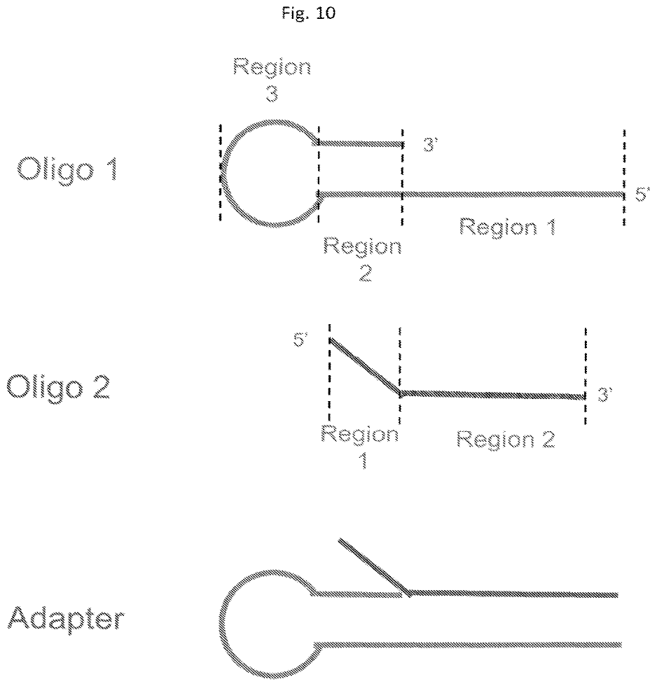

In certain embodiments, the adaptor comprises a first nucleic acid strand and a second nucleic acid strand. The first nucleic acid strand may comprise a 5′ segment and a 3′ segment. In some embodiments, 3′ segment of the first nucleic acid strand comprises a hairpin. The hairpin may comprise a loop and a self-complementary stem region. The 5′ segment of the first nucleic acid segment may comprise a sequence that is not self-complementary. Certain non-limiting examples of the adaptors are depicted in FIG. 8. For instance, the first nucleic acid strand 101 comprises a hairpin 102 and a 5′ segment that is not self-complementary 103.

The adaptor may comprise a second nucleic acid strand, wherein the second nucleic acid strand is non-contiguous with the first nucleic acid strand. The second nucleic acid strand may comprise a 3′ segment that is complementary with the 5′ segment of the first nucleic acid strand. In some embodiments, the second nucleic acid strand 104 may comprise a sequence 105 that is complementary to the 5′ segment of the first nucleic acid strand as depicted in FIG. X. The 5′ end of the adaptor molecule may comprise a region that blocks ligation of the 3′ end of the first nucleic acid strand to the 5′ end of the second nucleic acid strand. The region that blocks ligation may be a flap, a gap, a blocking group, or a combination thereof. The adaptor may comprise a gap 106 between the first nucleic acid strand 101 and the second nucleic acid strand 104. The second nucleic acid strand 104 may comprise a flap 107 that is not complementary to the first nucleic acid sequence. The second nucleic acid strand 104 may comprise a blocking group 108 at the 3′ end of the segment that is complementary to the 5′ segment of the first nucleic acid strand.

In some embodiments, described herein is an adaptor comprising: a first nucleic acid strand comprising: a 5′ segment of the first nucleic acid strand comprising a hairpin, and a 5′ segment of the first nucleic acid strand comprising a sequence that is not self-complementary; and a second nucleic acid strand comprising: a 3′ segment of the second nucleic acid strand comprising a sequence complementary to a portion of the 5′ segment of the first nucleic acid strand and a 5′ segment that blocks s ligation of the 3′ end of the first nucleic acid strand to the 5′ end of the second nucleic acid strand, thereby generating a non-contiguous segment between the 5′ end of the second nucleic acid strand and the 3′ end of the first nucleic acid strand, wherein the 5′ segment comprises a flap or a blocking group.

### 7. Blocking Groups

The adaptor molecule may comprise a blocking group designed to block ligation. The adaptor molecule may comprise blocking group designed to block extension. The blocking group may comprise a oligonucleotide. The oligonucleotide may comprise a locked nucleic acid, a psoralen modified nucleic acid, a MGB modified nucleic acid or G-quadruplex oligo. The blocking group may comprise a sequence that binds to a peptide or DNA binding protein. The peptide or protein may bind to the blocking sequence on the adaptor. The DNA binding protein may be a Cas protein. The DNA binding protein may be a Tus protein.

### 8. Sequencing Sites

The adaptor molecules may comprise a sequencing site. The adaptor molecules may comprise a primer hybridization site.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the term “overlaying” (e.g., overlaying images) refers to overlaying images from different cycles to generate a distribution of detected optical signals (e.g., position and intensity, or position of peak) from each analyte over a plurality of cycles. This distribution of detected optical signals can be generated by overlaying images, overlaying artificial processed images, or overlaying datasets comprising positional information. Thus, as used herein, the term “overlaying images” encompasses any of these mechanisms to generate a distribution of position information for optical signals from a single probe bound to a single analyte for each of a plurality of cycles.

A “cycle” is defined by completion of one or more passes and stripping of the detectable label from the substrate. Subsequent cycles of one or more passes per cycle can be performed. For the methods and systems described herein, multiple cycles are performed on a single substrate or sample. For DNA sequencing, multiple cycles requires the use of a reversible terminator and a removable detectable label from an incorporated nucleotide. For proteins, multiple cycles requires that the probe removal (stripping) conditions either maintain proteins folded in their proper configuration, or that the probes used are chosen to bind to peptide sequences so that the binding efficiency is independent of the protein fold configuration.

A “pass” in a detection assay refers to a process where a plurality of probes comprising a detectable label are introduced to the bound analytes, selective binding occurs between the probes and distinct target analytes, and a plurality of signals are detected from the detectable labels. A pass includes introduction of a set of antibodies that bind specifically to a target analyte. A pass can also include introduction of a set of labelled nucleotides for incorporation into the growing strand during sequencing by synthesis. There can be multiple passes of different sets of probes before the substrate is stripped of all detectable labels, or before the detectable label or reversible terminator is removed from an incorporated nucleotide during sequencing. In general, if four nucleotides are used during a pass, a cycle will only consist of a single pass for standard four nucleotide sequencing by synthesis.

As used herein, an image refers to an image of a field taken during a cycle or a pass within a cycle. In some embodiments, a single image is limited to detection of a single color of a detectable label.

As used herein, the term “field” refers to a single region of a substrate that is imaged. During a typical assay a single field is imaged at least once per cycle. For example, for a 20 cycle assay, with 4 colors, there can be 20*4=80 images, all of the same field.

A “target analyte” or “analyte” refers to a single molecule, compound, complex, substance or component that is to be identified, quantified, and otherwise characterized. A target analyte can comprise by way of example, but not limitation to, a single molecule (of any molecular size), a single biomolecule, a polypeptide, a protein (folded or unfolded), a polynucleotide molecule (RNA, cDNA, or DNA), a fragment thereof, a modified molecule thereof, such as a modified nucleic acid, or a combination thereof. In an embodiment, a target polynucleotide comprises a hybridized primer to facilitate sequencing by synthesis. The target analytes are recognized by probes, which can be used to sequence, identify, and quantify the target analytes using optical detection methods described herein.

A “probe” as used herein refers to a molecule that is capable of binding to other molecules (e.g., a complementary labelled nucleotide during sequencing by synthesis, polynucleotides, polypeptides or full-length proteins, etc.), cellular components or structures (lipids, cell walls, etc.), or cells for detecting or assessing the properties of the molecules, cellular components or structures, or cells. The probe comprises a structure or component that binds to the target analyte. In some embodiments, multiple probes may recognize different parts of the same target analyte. Examples of probes include, but are not limited to, a labelled reversible terminator nucleotide, an aptamer, an antibody, a polypeptide, an oligonucleotide (DNA, RNA), or any combination thereof. Antibodies, aptamers, oligonucleotide sequences and combinations thereof as probes are also described in detail below.

The probe can comprise a detectable label that is used to detect the binding of the probe to a target analyte. The probe can be directly or indirectly bound to, hybridized to, conjugated to, or covalently linked to the target analyte.

As used herein, the term detectable label refers to a molecule bound to a probe that is capable of generating a detectable optical signal when the probe is bound to a target analyte and imaged using an optical imaging system. The detectable label can be directly or indirectly bound to, hybridized to, conjugated to, or covalently linked to the probe. In some embodiments, the detectable label is a fluorescent molecule or a chemiluminescent molecule. The probe can be detected optically via the detectable label.

As used herein, the term optical distribution model refers to a statistical distribution of probabilities for light detection from a point source. These include, for example, a Gaussian distribution. The Gaussian distribution can be modified to include anticipated aberrations in detection to generate a point spread function as an optical distribution model.

The section headings used herein are for organizational purposes only and are not to be construed as limiting the subject matter described.

### IV. EXAMPLES

The following examples are included for illustrative purposes only and are not intended to limit the scope of the invention.

### Example 1: A Method of Library Preparation and Sequencing

This disclosure describes the creation of “DualSeq” libraries and sequencing, as well as a modality of reading methylation information from the libraries. Dual Seq allows for paired-end sequencing of an insert without any turnaround chemistry, and can enable sequencing both strands of a duplex in a paired-end manner. Any library can be used and ligated on looped adapters. A CAT is created by landing a primer on the open loop. Seq Readl and Read2. Physical (e.g. temperature), chemical (e.g. formamide) or enzymatic (e.g. helicases) means can be used to open secondary and tertiary structure for the sequencing primer to hybridize. A sequencing primer can be hybridized after loading. DNA polymerases with strand-displacement activity for efficient nucleotide incorporation can be used.

A DualSeq circle created by ligating 2 different adapters onto a double stranded DNA fragment of interest as depicted in FIG. 1. Adapters are asymmetrically ligated to enzymatically or sonically-sheared DNA. Mixtures of looped adapters can be ligated or various modalities can be used to select for inserts with two different adapters. Sequential ligation steps can be used to ligate two different adapters. Physical means such as beads in concert with sequential ligation steps can be used to ligate two different adapters. The circle in the case of DualSeq has two loop regions (L1, L2) that contain different primer hybridization sequences (P1,P2), as depicted in FIG. 2 The CAT molecule, as depicted in FIG. 3, obtained through rolling circle amplification of the circle in FIG. 2 will allow for the specific sequencing of both strands of the original dsDNA molecule via initiation at P1 or P2.

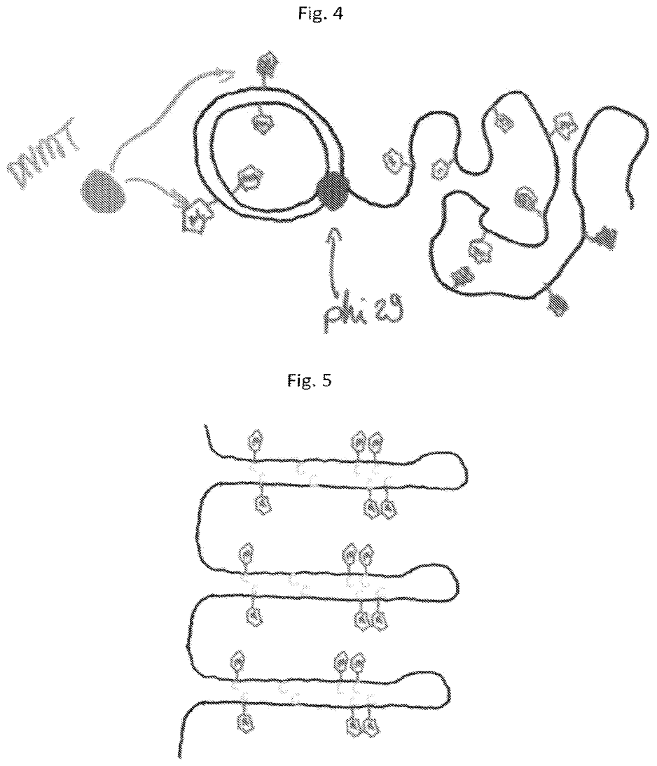

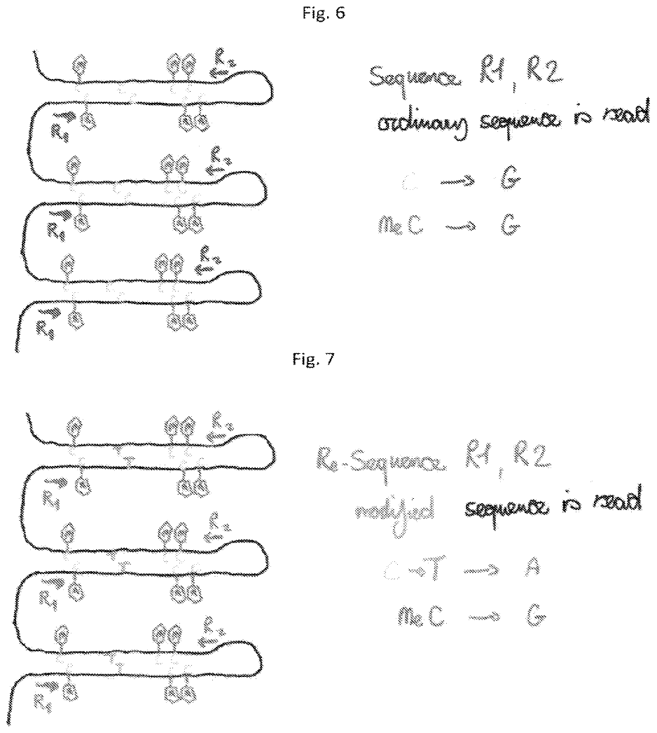

Methylation signature of circle DNA is preserved in CATs by performing rolling circle amplification (RCA) in the presence of DNA methyltransferase (DNMT), as depicted in FIG. 4. FIG. 5 depicts a DualSeq CAT with preserved methylation signatures. Methylated (blue) and unmethylated Cytosines (yellow) are shown. During the first rounds of sequencing the forward and reverse strands (RI, R2) are read, as depicted in FIG. 6. Both methylated and unmethylated cytosines are read through their complementary guanine (G) base pairing.

After the first round of sequencing, all unmethylated cytosines are enzymatically converted to thymine (T, green) as depicted in FIG. 7. A second round of sequencing of the forward and reverse strands (R1, R2) is performed, the methylated C's are still read via a G, while the newly converted T's are read via adenine (A) pairing.

### Example 2: Asymetric Sequencing and Library Generation

Two different adaptor sequences are ligated at both ends of DNA insert (asymmetric adaptor ligation) and a newly synthesized complementary strand is generated for a reference sequence in methyl sequencing application.

The workflow for achieving dual seq and dual seq based methyl seq using asymmetric ligation with clip adaptors is described below and depicted in FIG. 8.
1. Ligate Clip adaptor, which is designed to perform 3′ end extension from both end of the molecule.
2. The clip adaptor consists of two oligos hybridized together.
3. The ligated molecule will be extended from 3′ end using strand displacement DNA polymerase.
4. So, the newly synthesized complementary strand will serve as reference sequence while original strand carries methyl signature of each strand.
5. Ligate second adaptor at one double stranded open end.
6. Perform enzymatic conversation on the retained methylation information from the original strand where the unmethylated C will be converted to T leading to a G: T mismatch in the double stranded region of loop.
7. Amplify CAT using rolling circle amplification so that one strand will carry converted methyl signature and other strand will represent the reference sequence. This will create two different CATs.

The CAT from dual seq circle can potentially form a duplex structure, which can inhibit the sequencing chemistry. To facilitate the sequencing chemistry to work the following workflow can be used:
1. Hybridize a LNA/Psoralen/MGB modified, 3′ blocked oligos designed in the loop A specific region (as shown in blue in the figure).
2. Crosslink the psoralen modified oligos if we use cross-linking strategy.
3. Hybridize an oligo designed specific to loop B (as shown in green in the figure).
4. Perform primer extension from the B specific primers, which will extend and stop when it reaches to LNA/psoralen/MGB modified oligos previous hybridized.
5. This will create a triplex structure and the original complementary strand will become single stranded, and ready to hybridize for sequencing primer and sequencing.
6. Strip off the newly synthesized strand and sequenced strand using any denaturing agents like NaOH, formamide or enzymatically.
7. Repeat the process for other end of the loop after first round of sequencing and sequence complimentary strand from the other end.
8. If dUTP is used instead of dTTPs during triplex generation then USER or similar chemistry can be used to remove the triplex strand enzymatically and proceed to sequencing steps without using NaOH or formamide.
9. In this case the first sequencing primer may be extended to fill the strand until it reaches to the adaptor region.
10. This will give result in pair-end sequencing from both the strands and can be achieved methyl signature when applicable.

### Example 3: Generating Asymmetric Sequencing Libraries with Novel Adapters

DNA inserts are ligated to novel adapters, as depicted in FIG. 10. These adapters contain two independent oligonucleotides. Oligo 1 has 3 regions: Region 1 (5′ end) is single stranded; Region 2 is double stranded; and Region 3 is single stranded. Region 2 and 3 form a hairpin loop Oligo 2 has two regions: Region 1 (5′ end) is a single stranded flap and Region 2 (3′ end) is complementary to region 1 of oligo 1. An adapter is formed by hybridization of oligo 1 to oligo 2.

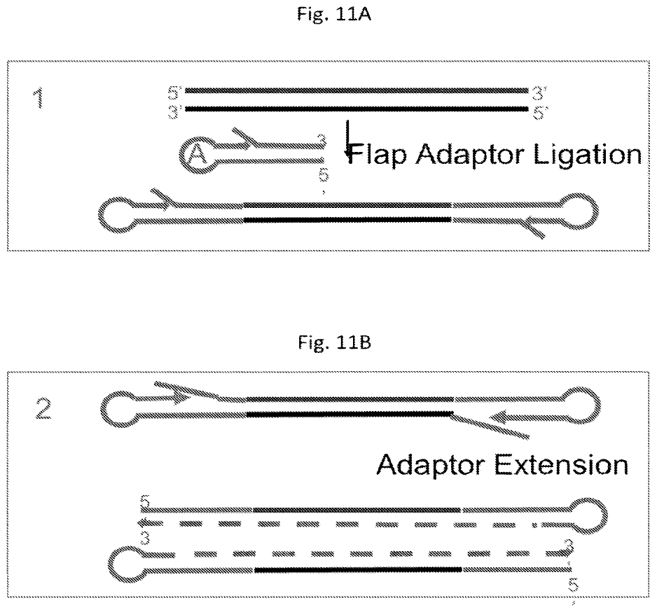

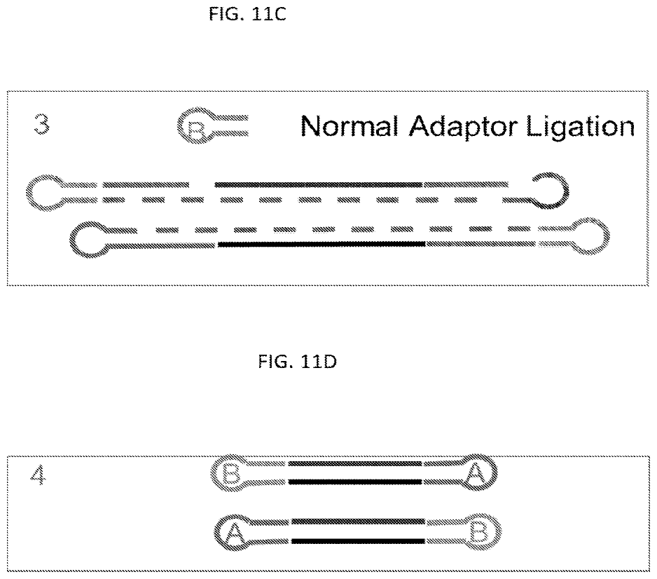

The adapters are ligated to DNA inserts to produce adapted DNA molecules with two identical adapters on the two ends (See FIG. 11A). The 3′ end of oligo 1 in each of the two adapters are extended in the 3′ to 5′ direction resulting in displacement of the flap (region 1 in oligo 2). The extending DNA strand (dotted line) displaces the flap and the attached DNA strand.

Extension produces a pair of double stranded DNA molecules with a stem loop adapter on one end and an open adapter of the other end, as depicted in FIG. 11B.

A normal stem loop adapter with a different sequence then the flap adapter is then ligated to the open adapter end, as depicted in FIG. 11C. This results in in adapted DNA molecules with two different adapters on each end, i.e., an asymmetric library with adapter A and adapter B, as depicted in FIG. 11D.

### Example 4: Paired-End Sequencing from Asymetric Libraries

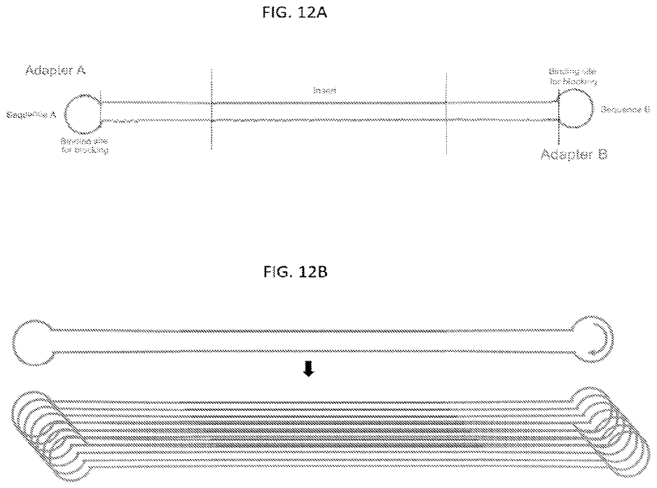

Asymetric libraries, as depicted in FIG. 12A, are generated used the methods described herein. The asymmetric libraries are comprised of 2 different adapters. Each adaptor has a different sequence in the loop region. The loop region contains a site for a blocking reagent to bind. The blocking reagent may be a Locked Nucleic Acid or a sequence specific binding protein such as dCas9. An extension primer binds to the asymmetric library and is extended by Rolling Circle Amplification producing a concatemer that binds with itself creating a double stranded accordion like structure since both the forward and reverse complement strands are present, as depicted in FIG. 12B.

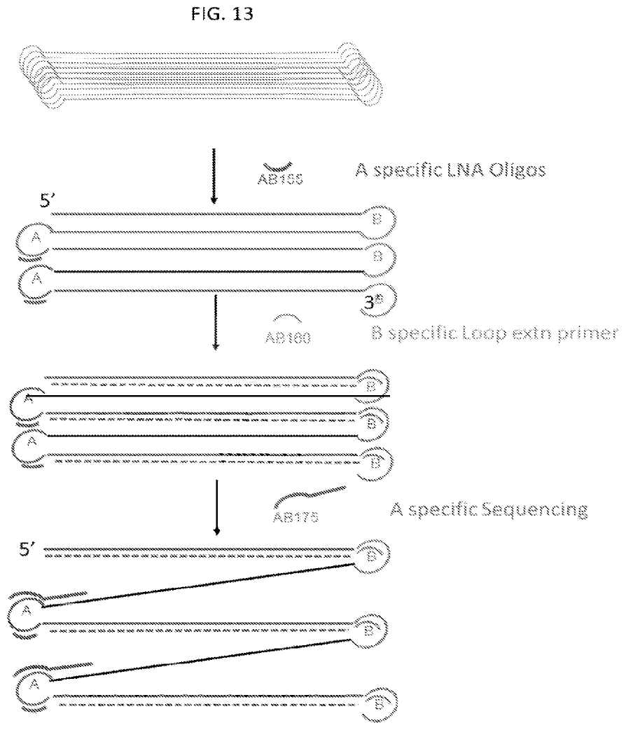

Paired end sequencing of the first strand from adapter A comprises 4 steps, as depicted in FIG. 13. A blocking agent specifically attaches to the said blocking region of adapter A. In the example shown, the blocking reagent is a Locked Nucleic Acid. The LNA binds to the blocking site on stem loop A only. An extension primer that binds to a site on stem loop B only. The extension primer is extended till it reaches the blocking agent forming a double stranded strand (solid and dotted line). The bottom strand is now single stranded and can hybridize to a sequencing primer. A sequencing primer that binds to a site on stem loop A only is hybridized. The sequencing primer is then extended by a sequencing polymerase in the process determining the sequence of bases on the bottom strand.

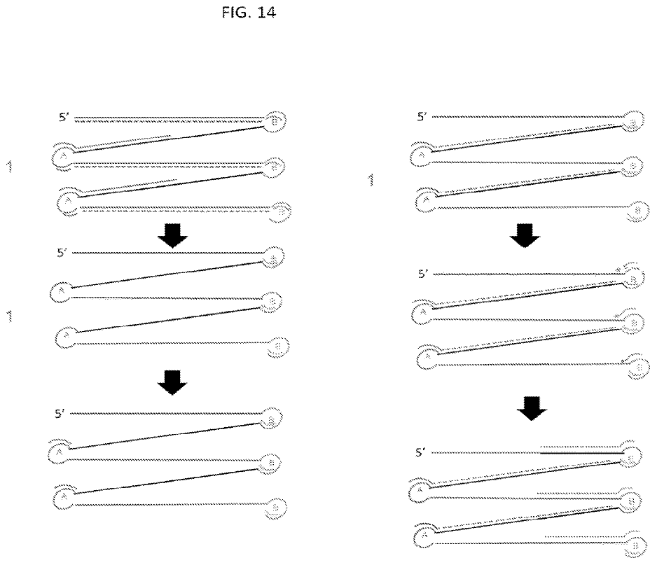

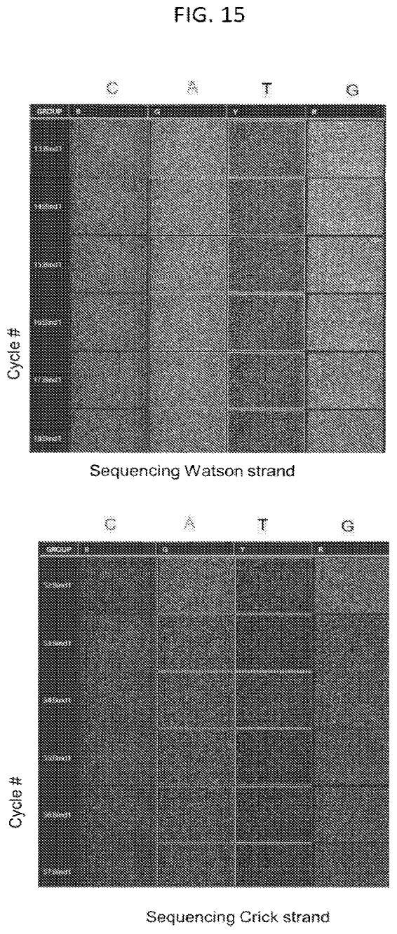

Next, sequencing of the second strand from adaptor B occurs, as depicted in FIG. 14. A denaturing reagent is used to removed the blocking agent, the sequenced strand and the previously extended strand from the previous sequencing reactions. A blocking agent that specifically attaches to the said blocking region of adapter A. In the example shown, the blocking reagent is a Locked Nucleic Acid. The LNA binds to the blocking site on stem loop A only. An extension primer that binds to a site on stem loop B only. The extension primer is extended till it reaches the blocking agent forming a double stranded strand (solid and dotted line). The bottom strand is now single stranded and can hybridize to a sequencing primer. A sequencing primer that binds to a site on stem loop A only is hybridized. The sequencing primer is then extended by a sequencing polymerase in the process determining the sequence of bases on the bottom strand.

### Example 5: Paired End Sequencing of the First and Second Strand

Paired end sequencing of asymmetric libraries was performed as described herein. The results are depicted in FIGS. 15-17. FIG. 15 depicts images of the sequencing surface during sequencing. Each row is one sequencing cucle. The columns are the fluorescence for each cycle separated into individual images representing each nucleotide. This demonstrates that both the forward and reverse strands were successfully sequenced.

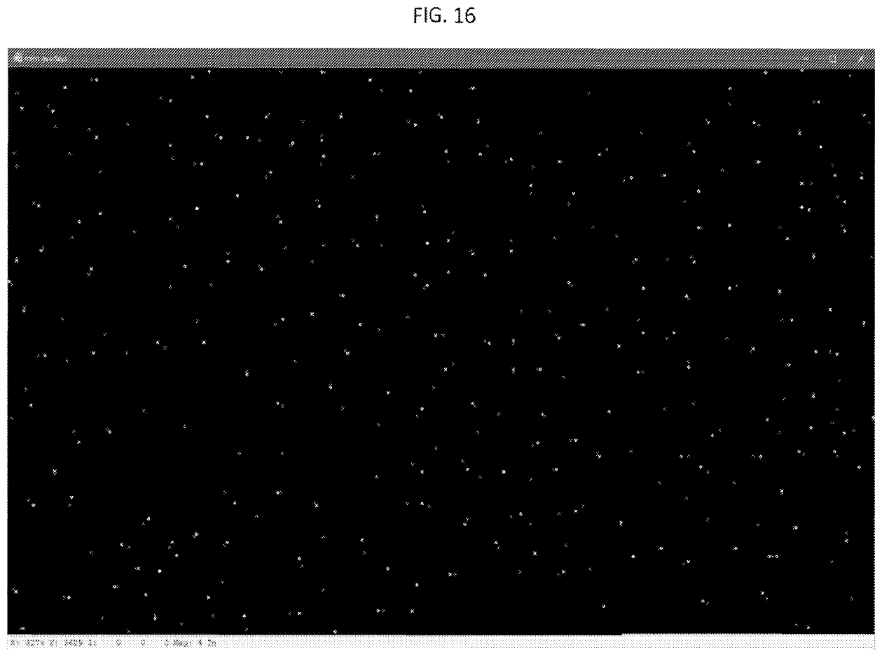

FIG. 16 depicts the location of the sequencing reads from the first strand (red) and the second strand (green). This indicates that the molecules were successfully attached and identified for forward and reverse sequencing.

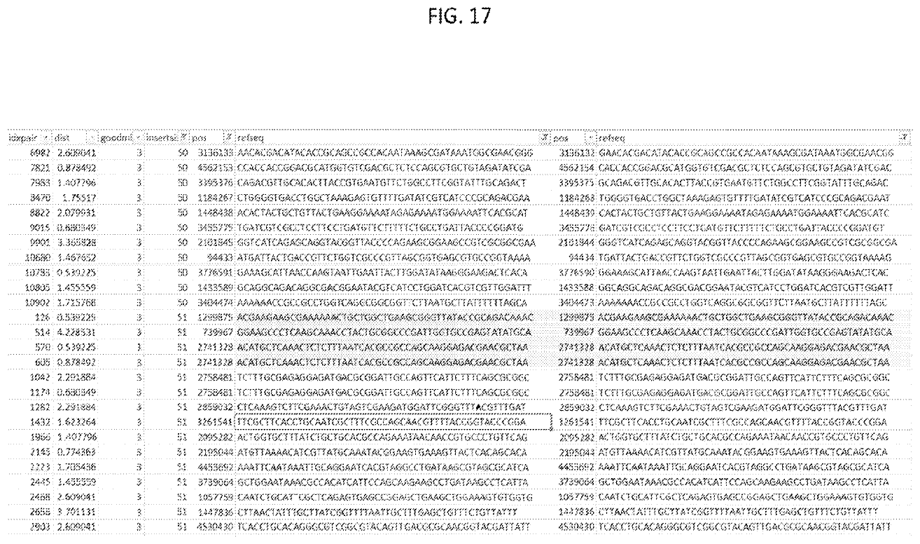

FIG. 17 shows base calls from the first and second strand. The sequence generated from the amplified concatamers are shown as base calls. The first column of basecalls were from the first strand and the second column of base calls were the reverse complement base calls from the second strand. The sequences were identical, showing high accuracy between forward and reverse sequencing.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of preparing a double-stranded sample nucleic acid for sequencing, wherein the double-stranded sample nucleic acid comprises a forward sample strand and a reverse sample strand, the method comprising:
(a) contacting the sample nucleic acid with one or more first adaptors to form a sample-adaptor complex, wherein the one or more first adaptors prevent ligation between the 5′ end of the forward sample strand and the 5′ end of the reverse sample strand;
(b) extending with a polymerase to form a forward double-stranded sample-adaptor complex comprising the forward strand, the first adaptor, and a sequence complementary to the forward strand and a reverse double-stranded sample-adaptor complex comprising the reverse sample strand, the first adaptor and a sequence complementary to the reverse sample strand; and
(c) ligating one or more second adaptors to the forward double-stranded sample-adaptor complex and the reverse double-stranded sample-adaptor complex to form a circular forward-adaptor complex and a circular reverse-adaptor complex.

2. The method of claim 1, wherein the forward double-stranded sample-adaptor complex and the reverse double-stranded sample-adaptor complex produced in step (b) comprise a blunt end and a looped end.

3. The method of claim 1, wherein the first adaptor comprises a first primer hybridization site, and the second adaptor comprises a second primer hybridization site.

4. The method of claim 3, wherein the first primer hybridization site and the second primer hybridization site are different.

5. The method of claim 1, wherein the first adaptor and the second adaptor are different.

6. The method of claim 1, wherein the first adaptor comprises:
(a) a first nucleic acid strand comprising:
i. a 3′ segment of the first nucleic acid strand comprising a self-complementary stem and a loop, and
ii. a 5′ segment of the first nucleic acid strand comprising a sequence that is not self-complementary; and
(b) a second nucleic acid strand comprising:,
i. a 3′ segment of the second nucleic acid strand comprising a sequence complementary to a portion of the 5′ segment of the first nucleic acid strand and
ii. a 5′ segment that blocks s ligation of the 3′ end of the first nucleic acid strand to the 5′ end of the second nucleic acid strand, thereby generating a non-contiguous segment between the 5′ end of the second nucleic acid strand and the 3′ end of the first nucleic acid strand, wherein the 5′ segment comprises a flap or a blocking group.

7. The method of claim 6, wherein the first adaptor prevents ligation by a gap, a flap, or a blocking group.

8. The method of claim 1, wherein the second adaptor comprises a hairpin.

9. The method of claim 1, further comprising contacting the circular forward-adaptor complex or the circular reverse-adaptor complex with a methyltransferase enzyme.

10. A method, comprising:
(a) providing a double-stranded nucleic acid molecule comprising a first strand and a second strand hybridized to the first strand;
(b) ligating adaptors to ends of the double-stranded nucleic acid molecule to yield a circular nucleic acid molecule; and
(c) using the circular nucleic acid molecule as a template to generate a single-stranded nucleic acid molecule comprising copies of the first strand and the second strand.

11. The method of claim 10, wherein the a first adaptor is ligated to a first end of the double-stranded nucleic acid molecule before a second adaptor is ligated to a second end of the double-stranded nucleic acid molecule.

12. The method of claim 11, wherein the double-stranded nucleic acid molecule is immobilized when the first adaptor is ligated to the first end of the double-stranded nucleic acid molecule.

13. The method of claim 10, wherein a first adaptor and a second adaptor are simulatanously ligated.

14. The method of claim 13, further comprising selecting a circular nucleic acid molecule wherein the first adaptor and the second adaptor are different.

15. The method of claim 10, wherein step (b) comprises:
(i) ligating Y-adaptors to ends of the double-stranded nucleic acid molecule to produce an adaptor-nucleic acid complex;
(ii) amplifying the adaptor-nucleic acid complex to produce a nucleic acid complex with a first adaptor sequence at a first end and a second adaptor sequence at a second end; and
(iii) ligating the 5′ end of the first adaptor sequence with the 3′ end of the first adaptor sequence and ligating the 5′ end of the second adaptor sequence with the 3′ end of the second adaptor sequence to produce a circule nucleic acid molecule.

16. The method of claim 10, wherein the first adaptor comprises a first primer hybridization site, and the second adaptor comprises a second primer hybridization site.

17. The method of claim 10, wherein the first primer hybridization site and the second primer hybridization site are different.

18. The method of claim 10, further comprising contacting the single-stranded nucleic acid molecule with a methyltransferase enzyme.

19. A method of paired end clonal amplification comprising:
(a) contacting a sample double-stranded DNA molecule comprising a template sequence with a first adaptor comprising a first adaptor sequence and a second adaptor comprising a second adaptor sequence to produce a circular DNA molecule, wherein the first adaptor sequence comprises a first primer hybridization sequence and wherein the second adaptor sequence comprises a second primer hybridization sequence;
(b) contacting the circular DNA molecule with a polymerizing enzyme to produce a single-stranded DNA molecule via rolling circle amplification, wherein the single-stranded DNA molecule comprises a sequence comprising at least the first adaptor sequence, the template sequence, the second adaptor sequence, and a sequence complementary to the template sequence; and
(c) subjecting the single-stranded DNA molecule to clonal amplification from at least (i) the first primer hybridization sequence and (ii) the second primer hybridization sequence.

20. The method of claim 17, wherein subjecting the single stranded DNA molecule to clonal amplification from (i) the first primer hybridization sequence comprises contacting the ssDNA with a blocking molecule that prevents extension of a nascent sequence beyond the second adaptor; wherein the nascent sequence is complementary to the template sequence or the sequence complementary to the template sequence.

21. The method of claim 17, wherein subjecting the single stranded DNA molecule to clonal amplification from (ii) the second primer hybridization sequence comprises contacting the ssDNA with a blocking molecule that prevents extension of a nascent sequence beyond the first adaptor; wherein the nascent sequence is complementary to the template sequence or the sequence complementary to the template sequence.

22. The method of claim 17, wherein the blocking molecule comprises an oligonucleotide.

23. The method of claim 22, wherein the oligonucleotide comprises a locked nucleic acid (LNA), a psoralen modified nucleic acid, a MGB modified nucleic acid, or a G-quadruplex oligo.

24. The method of claim 17, wherein the blocking molecule comprises a peptide.

25. The method of claim 24, wherein the peptide comprises a sequence-specific DNA binding protein.

26. The method of claim 25, wherein the sequence-specific DNA binding protein is a Cas protein or a Tus protein.

27. The method of claim 17, wherein the clonal amplification in (c)(i) is performed before the clonal amplification in (c)(ii).

28. The method of claim 17, wherein the clonal amplification in (c)(ii) is performed before the clonal amplification in (c)(i).

29. The method of claim 17, wherein the single-stranded DNA molecule further comprises a second copy of the first adaptor sequence, a second copy of the sequence of interest, a second copy of the second adaptor sequence, and a second sequence complementary to the sequence of interest.

30. The method of claim 17, wherein step (a) occurs in solution.

31. The method of claim 17, wherein step (b) occurs in solution.

32. The method of claim 17, wherein step (c) occurs in a solid state.

33. The method of claim 17, wherein the first adaptor is ligated to the double-stranded DNA molecule before the second adaptor is ligated to the double-stranded DNA molecule.

34. The method of claim 17, wherein the first adaptor comprises:
(a) a first nucleic acid strand comprising:
i. a 3′ segment of the first nucleic acid strand comprising a self-complementary stem and a loop, and
ii. a 5′ segment of the first nucleic acid strand comprising a sequence that is not self-complementary; and
(b) a second nucleic acid strand comprising:,
i. a 3′ segment of the second nucleic acid strand comprising a sequence complementary to a portion of the 5′ segment of the first nucleic acid strand and
ii. a 5′ segment that blocks s ligation of the 3′ end of the first nucleic acid strand to the 5′ end of the second nucleic acid strand, thereby generating a non-contiguous segment between the 5′ end of the second nucleic acid strand and the 3′ end of the first nucleic acid strand, wherein the 5′ segment comprises a flap or a blocking group.

35. The method of claim 17, wherein the circular DNA molecule comprises either the forward strand of the sample double-stranded DNA molecule or the reverse strand of the sample double-stranded DNA molecule, but not both.

36. The method of claim 17, wherein the first adaptor and the second adaptor are ligated to the double-stranded DNA molecule simultaneously.

37. The method of claim 17, wherein the circular DNA molecule comprises the forward strand of the sample double-stranded DNA molecule and the reverse strand of the sample double-stranded DNA molecule.

38. An adaptor, comprising:
(a) a first nucleic acid strand comprising:
i. a 3′ segment of the first nucleic acid strand comprising a self-complementary stem and a loop, and
ii. a 5′ segment of the first nucleic acid strand comprising a sequence that is not self-complementary; and
(b) a second nucleic acid strand comprising:,
i. a 3′ segment of the second nucleic acid strand comprising a sequence complementary to a portion of the 5′ segment of the first nucleic acid strand and
ii. a 5′ segment that blocks ligation of the 3′ end of the first nucleic acid strand to the 5′ end of the second nucleic acid strand, thereby generating a non-contiguous segment between the 5′ end of the second nucleic acid strand and the 3′ end of the first nucleic acid strand, wherein the 5′ segment comprises a flap or a blocking group.

39. The adaptor of claim 38, wherein the adaptor comprises DNA.

40. The adaptor of claim 38, wherein the adaptor comprises a first sequencing site.

41. The adaptor of claim 38, wherein the 5′ segment of the second nucleic acid comprises a flap of greater or equal to 1 nucleotides.

42. The adaptor of claim 38, wherein the 5′ segment of the second nucleic acid comprises a blocking group.

43. The adaptor of claim 42, wherein the blocking group is an oligonucleotide or a sequence for binding a sequence-specific DNA binding protein.

44. The adaptor of claim 43, wherein the oligonucleotide comprises a locked nucleic acid (LNA), a psoralen modified nucleic acid, a MGB modified nucleic acid, or a G-quadruplex oligo.

45. The adaptor of claim 43, wherein the sequence-specific DNA binding protein comprises a Cas protein or a Tus protein.

46. A DNA molecule comprising:
(d) a first DNA strand comprising:
i. a first segment comprising a first sequence; wherein the first segment is ligated to a first hairpin segment comprising a first hairpin sequence at the 3′ end of the first hairpin segment;
ii. a second segment comprising a second sequence that is complementary to the first sequence; wherein the second segment is ligated to the first hairpin segment at the 5′ end of the second segment and wherein the second segment is ligated to a second hairpin segment comprising a second hairpin sequence at the 3′ end of the second segment;
iii. a third segment comprising the first sequence, wherein the third segment is ligated to the second hairpin sequence at the 5′ end of the third segment and wherein the third segment is ligated to a third hairpin segment comprising the first hairpin sequence at the 3′ end;
iv. a fourth segment comprising the second sequence; wherein the fourth segment is ligated to the third hairpin segment at the 5′ end of the fourth segment; and
(e) a second DNA strand comprising a sequence complementary to the first sequence.
